(19) United States
(12) Patent Application Publication
(10) Pub. No.: US 2019/0298253 A1
(43) Pub. Date: Oct. 3, 2019

(54) JOINT DISORDER DIAGNOSIS WITH 3D MOTION CAPTURE

(71) Applicant: Baylor Research Institute, Dallas, TX (US)

(72) Inventor: Martin D. HAL, Dallas, TX (US)

(21) Appl. No.: 16/068,048

(22) PCT Filed: Jan. 27, 2017
(86) PCT No.: PCT/US17/15430

§ 371 Date: Jul. 3, 2018

Related U.S. Application Data
(60) Provisional application No. 62/289,033, filed on Jan. 29, 2016.

(51) Int. Cl. A61B 5/00; A61B 5/11; G16H 50/20

(52) U.S. Cl. CPC A61B 5/4528; A61B 5/1121; A61B 5/6804; G16H 40/63; A61B 5/4824; G16H 50/20; A61B 5/002; A61B 5/1128

(57) ABSTRACT

A system for diagnosing a patient's joint disorder. A patient is fitted with a body suit, which can comprise one or more straps, sleeves, or the like. Sensors in the suit are placed around a problematic joint. The patient executes a number of selected ranges of motion, or tests and transmits signals indicative of pain level at different positions. A computer system receives the captured motion data and pain data and compares same to stored data sets. The stored data sets may comprise other motion data and/or pain data from the patient being examined, or captured data from other patients. The stored data sets can also be tagged with, or otherwise associated with, a diagnosis, model can be formed with respect to the patient's captured motion and pain information. A probable diagnosis of the joint disorder, effectiveness of the patient's treatment regimen, and disorder progression can be ascertained.

### CROSS-REFERENCE TO RELATED APPLICATIONS

This application claims the benefit of U.S. Provisional Patent Application No. 62/110,265 entitled, “JOINT DISORDER DIAGNOSIS WITH 3D MOTION CAPTURE” filed on Jan. 30, 2015; and of U.S. Provisional Patent Application No. 62/289,033 entitled, “JOINT DISORDER DIAGNOSIS WITH 3D MOTION CAPTURE” filed on Jan. 29, 2016, both of which are expressly incorporated by reference herein in their entireties.

### TECHNICAL FIELD

The present disclosure relates to medical diagnostic technology. Specifically, the present disclosure relates to a three-dimensional motion capture system used to improve consistency in diagnosing joint disorders.

### BACKGROUND

The diagnosis and treatment of joint disorders in the human body is a rapidly evolving discipline that integrates multiple fields of study. Recent technological advancements in imaging a problematic joint allow physicians and therapists to discover potential diagnoses “below the surface.” Nevertheless, a thorough physical examination of a patient's joint is critical in assessing joint pathology. These physical examinations often require, among other things, that a patient move body parts comprising a problematic joint through different ranges of motion or undergo particular tests.

For example, with respect to the hip joint, physical examinations have progressed from clinical evaluation of muscle contracture to more sophisticated measures including dynamic movement, neurovascular assessment, and proprioception. Although there is a consensus among hip surgeons regarding the most effective physical examination maneuvers, proper interpretation of examination results requires a steep learning curve and extensive time and training. Also, dynamic tests that assess range of motion or tests often lack consistency, especially among younger or less experienced surgeons. Accordingly, there is a need for a system that improves consistency in diagnosing joint disorders while reducing the time and training required to make a proper diagnosis.

### SUMMARY

In view of the foregoing, according to concepts described herein, a human joint diagnostic method includes capturing, using sensors attached to a test patient, positions for one or more preselected points of interest of the test patient through a preselected range of motion or test. The method also includes receiving, at one or more processors in communication with the sensors, the captured positions and a pain value associated with the captured positions. The method further includes compiling, with the one or more processors, the captured positions and the pain values associated with the captured positions to generate a test patient profile. Also, the method includes comparing, using one or more processors, the test patient profile to one or more stored profiles. The one or more stored profiles comprise positions for the one or more preselected points of interest of another test patient through the preselected range of motion or test and a pain value associated with the positions. Each stored profile is associated with a diagnosis. The method further includes, based on the comparing, associating the test patient profile with a diagnosis associated with the one or more stored profiles that matches the test patient profile.

In other aspects, a human joint diagnostic system includes sensors to capture positions for one or more preselected points of interest of a test patient through a preselected range of motion or test. The system also includes one or more processors in communication with the sensors, the one or more processors configured to receive the captured positions and pain values associated with the captured positions. The processor is also configured to compile the captured positions and the pain values associated with the captured positions to generate a test patient profile. The processor is also configured to compare the test patient profile to one or more stored profiles, the one or more stored profiles comprising positions for the one or more preselected points of interest of another test patient through the preselected range of motion or test and pain values associated with the positions, each stored profile associated with a diagnosis. The processor is also configured to, based on the comparing, associate the test patient profile with a diagnosis associated with the one or more stored profiles that matches the test patient profile.

The foregoing has outlined rather broadly the features and technical advantages of the present invention in order that the detailed description of the invention that follows can be better understood. Additional features and advantages of the invention will be described hereinafter which form claims of the invention. It should be appreciated by those skilled in the art that the conception and specific embodiments disclosed can be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### DESCRIPTION OF THE FIGURES

For a more complete understanding of the concepts described herein, reference is now made to the following descriptions taken in conjunction with the accompanying drawings, in which:

FIG. 1 illustrates a joint disorder diagnosis system according to an embodiment;

FIG. 2 illustrates a joint disorder method according to an embodiment;

FIG. 3 illustrates a joint disorder method according to another embodiment; and

FIG. 4 illustrates a joint disorder diagnostic module according to an embodiment.

### DETAILED DESCRIPTION

In view of the foregoing, embodiments described herein provide a system for improving the diagnosis of a patient's joint disorder. A patient can be fitted with straps or attachments that support sensors placed about a problematic joint, or with a body suit that comprises a number of sensors placed around the problematic joint. The patient executes a number of selected ranges of motion or tests. While doing so, the patient transmits signals indicative of his or her pain level at different positions in the ranges of motion or tests. A computer system receives the captured motion data and pain data and compares same to stored data sets. The stored data sets may comprise other motion data and/or pain data from the patient being examined, or captured data from other patients. The stored data sets can also be tagged with, or otherwise associated with, a diagnosis. Based upon the comparison, a model can be formed with respect to the patient's captured motion and pain information. Meaningful information, such as a probable diagnosis of the joint disorder, effectiveness of the patient's treatment regimen, and progression of the joint disorder itself can be ascertained from the comparison.

Joint disorders or injuries often occur while moving the joint, or at least are most perceptible during movement of the joint. As such, accurate diagnosis of disorders that occur while moving, or are most perceptible while moving, requires a diagnostic system that actually analyzes joint movement. As such, described embodiments are optimal for diagnosing such disorders because they provide a system that enables one to visualize, analyze, and provide diagnostic data while a patient is in motion. This can be done in real-time and while the patient executes selected activates that are thought to enable an accurate diagnosis. These activities may involve selected ranges of motion or tests as well as common passive and/or active activities that are meant to support a diagnostic protocol. Examples of active activities include, without limitation, walking, running, turning, bending, or lifting. Examples of a passive activity may include a passive range of motion or test.

Embodiments provide real-time diagnostic images of muscular and skeletal function and dysfunction of the human body in motion. Embodiments also display accurate biomechanics on a patient-by-patient basis by adjusting to the specific measurements and morphology of each patient. This data can then give healthcare providers information needed to quantify specific injuries and dysfunctions and accurately and objectively apply diagnostic and treatment protocols. Accordingly, described embodiments will improve the diagnosis of joint disorders. These improvements will reduce health care costs by allowing for more accurate and objective descriptions of joint pathologies and standardizing the physical examination of joint disorders across different levels of healthcare providers.

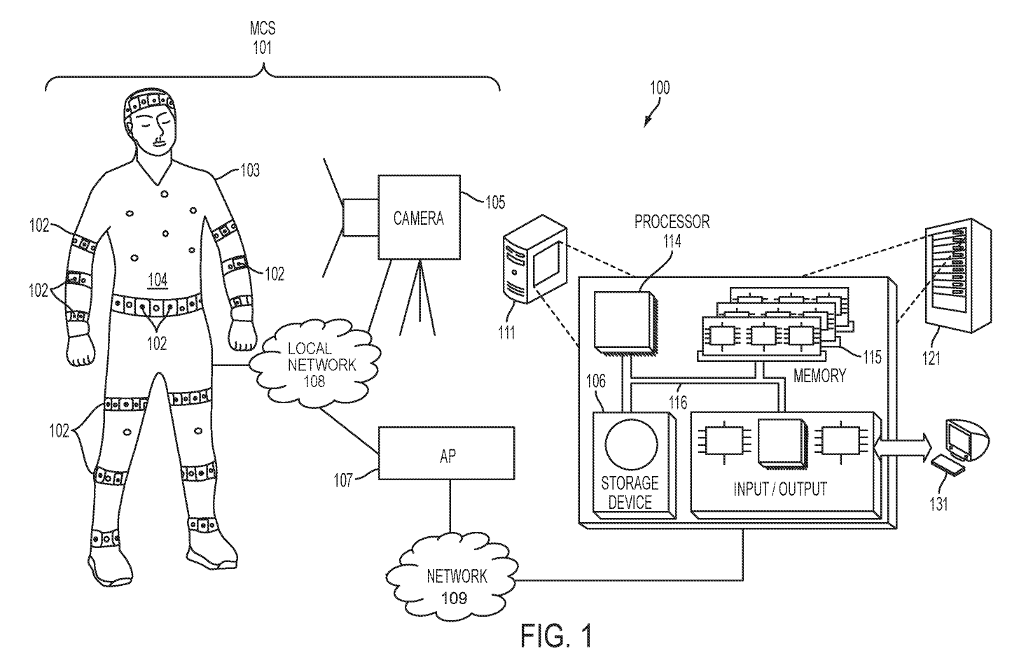

FIG. 1 illustrates an embodiment of joint disorder diagnosis system 100. The illustrated embodiment comprises motion capture system 101, diagnostic computer 111, database 121, and graphical user interface 131. The components of system 100 can be implemented at a central facility to aggregate diagnostic data and offer services to users of system 100, or distributed across one or more networks to, e.g., improve capacity and data load balancing across system 100 and associated networks.

Motion capture system (MCS) 101 includes sensors 102 placed on a motion capture device implemented as a specifically designed motion capture suit 103. Different types of sensors 102 can be placed around joints of the body to provide the user with specific data showing the movement and position of bones and muscles comprising a problematic joint. Suit 103 can be made in various sizes and have multiple adjustments to accommodate a sufficient fit for patients of various sizes and shapes. Suit 103 may be customized to optimize its fit around varying body parts, including shoulders, elbows, hips, hands, feet, and the like, of different sizes. Suite 103 can also comprise one or more straps or modular attachments that support sensors 102 about the problematic joint(s). In this way, a patient is not required to fit into an entire suit, but can simply secure the straps about the joint. Either way, suit 103 can be made of a comfortable and flexible material to facilitate unencumbered motion by patient 104 during analysis. In some embodiments, body suit 103 comprises a neoprene material like a foamed neoprene, nylon backed neoprene, or lycra backed neoprene.

Portions of body suit 103 can be implemented as cylindrical bands that wrap around patient 104 at particular locations, such as one or more joints of patient 104. Otherwise, a particular body suit 103 can be formed to fit a certain joint, such as a hip joint. In such case, the shape of body suit 103 will not necessarily be cylindrical, but will conform to the shape of the given joint such that suit 103 encompasses at least a reference or measurement point for sensors 102 surrounding the problematic joint. Each body suit 103 can be flexed to a certain extent, depending upon the desired or permitted range of motion, or test. Again, where suit 103 comprises one or more straps or similar attachment means (such as, e.g., a sleeve), the straps can simply be wrapped around the problematic joint(s). This may be preferred so that sensors 102 can be placed upon, and moved about, the patient more quickly.

Preferably, sensors 102 are attached to body suit 103 so that adjacent sensors 102 are separated by a fixed or semi-fixed distance from each other when measured along an axis of body suit 103 that passes through sensors 102. Pairs of adjacent sensors 102, in turn, can be separated by the same distance. Accordingly, a number of sensors 102 can be formed on body suit 103 so that sensors 102 are evenly spaced apart. In some embodiments, however, sensors 102 are not required to be evenly spaced apart, but can maintain some distance from one another that does not substantially change.

Sensors 102 can be planar with respect to body suit 103, can be non-planar (e.g., protrude) with respect to body suit 103, or a combination thereof. In FIG. 1, each sensor 102 is implemented with a dot that lies in the same plane as body suit 103. The same hold true when suit 103 merely comprises straps or sleeves that include one or more sensors 102.

Sensors 102 can have any of one or more geometric shapes. For example, sensors 102 can be implemented with circles, triangles, squares, or rectangles. As shown in FIG. 1, each sensor 102 has the same geometric shape. Sensors 102 can have one or more colors, and preferably colors that sharply contrast with the colors of body suit 103. Further, sensors 102 can be covered with or composed of a luminous material, or can be self-illuminating, such as sensors 102 that incorporate light emitting diodes (LEDs). For example, infra-red, self-illuminating sensors 102 can be seen when in dark environments, and, as a result, provide invariance to lighting conditions. Therefore, luminous or illuminated sensors 102 may facilitate performing motion capture when patient 104 is in either a light or dark environment.

It should be appreciated that a variety of systems can be used to detect the motion of the suit worn by patient 104. In some embodiments, optical motion capture devices can be used to capture the motion of the patient's body. Such detection systems comprise passive sensors that deflect light and/or active sensors that emit light (e.g., LED light, infrared, or some other detectable signal). The active sensors can be modulated with respect to time in order to facilitate accurate detection, e.g., by having different sensors transmit light or other signals on a periodic basis to avoid undue interference. In some embodiments, no special sensors are placed on suit 103. Rather, the optical detection device directly focuses on the body alone.

In some embodiments, sensors 102 can be implemented with coded indicia, e.g., a bar code, QR code, or the like. One advantage of using sensors 102 with coded indicia is that the indicia can be used to uniquely identify each sensor 102. For example, a computer in communication with sensors 102 (e.g., diagnostic computer 111) may include an index that correlates observed coded indicia with particular sensors 102. When the indicia is captured by a camera, the index can be accessed to identify which sensor 102 is specified by the coded indicia (e.g., the identified sensor can be a sensor placed along the outer portion of the right hip).

In one embodiment MCS 101 is an optical motion capture system and includes one or more cameras 105. Such an embodiment uses sensors 102, whether reflective or light-emitting, to generate a series of points which are filmed by cameras 105. Several of cameras 105 can be used to provide a 3D assessment by mapping multiple sensor 102 points onto a computer model executed at, e.g., diagnostic computer 111. A related embodiment may even include sensor-less arrangements where a computer, such as diagnostic computer 111, analyzes multiple streams of optical input received from cameras 105 and identifies the form and motion of patient 104.

Cameras 105 may include or be connected to storage medium 106 to digitally record the captured images, as well as telemetry and transceiver software and hardware to transmit data structures comprising image data and other information to diagnostic computer 111. This enables MCS 101 and other system components to wirelessly communicate with one another. As such, additional information including updates, control data, system performance and diagnostic data, and the like, may be communicated between system components. This is particularly advantageous for embodiments where system 100 is distributed, where, e.g., portions of diagnostic computer 111 reside on one or more mobile devices.

In operation, one or more body suits 103 are placed around the body of patient 104 to capture the skeletal motion of patient 104. Any number of sensors 102 located on body suit 103, including zero, can be seen by each camera 105. For example, body suit 103 can have three sensors 102 that are fully visible from each camera view. In another example, only a single sensor 102 can be seen in a camera view. In some embodiments, none of cameras 105 need to see the same sensors 102 to reconstruct the motion of patient 104, as cameras 105 can record the motion of patient 104's body, which is fed into modeling software or the like executing on diagnostic computer 111.

In another embodiment MCS 101 is a non-optical motion capture system. Accordingly, sensors 102 are capable of measuring their own absolute and relative position and may comprise inertial systems such as accelerometers, gyroscopes, and the like, as well as data inputs and data outputs. According to such an embodiment, sensors 102 transmit position data to other system components (e.g., access point 107 or camera 105), where the data is recorded and may be processed to meaningfully interpret the data. As known in the art, in non-optical embodiments, camera 105 essentially functions as a wireless data transceiver, operating to receive the non-optical data structures and process them accordingly.

In embodiments where MCS 101 comprises a non-optical motion capture system, sensors 102 can be placed in appropriate positions on suit 103 and transmit a signal indicative of their position. For example, inertial motion and/or oscillation sensors can transmit coordinates to modeling software and hardware residing at diagnostic computer 111. The transmissions can be performed wirelessly to allow patient 104's movement to be unencumbered by wiring. Accordingly, each sensor 102 comprises a three-axis accelerometer, and a three-axis gyroscope array such as a dual-axis gyroscope and a single-axis gyroscope.

To promote comfort while minimizing resistance attributable to suit 103, sensors 102 may be implemented in suit 103 as a series of electromagnetic fibers sewn into suit 103. Naturally, these features extend to cases where suit 103 comprises an entire suit fitted on patient 104, or merely one or more straps or other attachment means placed only about one or more joints of patient 104. In such case, each sensor 102 may actively transmit data to other system components. Otherwise, sensors 102 may be passive, where signals periodically transmitted from, e.g., camera or transceiver 105 are reflected from sensors 102 throughout a range of motion, or test. The reflected signals are captured at camera or transceiver 105 throughout the range of motion, or test for further processing.

Sensors 102 may comprise hardware and software enabling other properties associated with patient 104 to be sensed and communicated to other system components. In one embodiment, one or more sensors 102 may comprise a flexion sensor which can be applied around a joint to determine the extent of bending. Sensors 102 may also comprise a pressure sensor and/or a temperature sensor. Similarly, magnetic sensors can be used to transmit motion and/or position information. Each sensor 102 may further include a vibration transducer which can be activated by a local processor. The vibration transducer can be used to provide a sensory input at a certain physical position or orientations of sensors. For example, the vibration transducer can vibrate when patient 104 adopts an incorrect posture or incorrectly executes a predetermined range of motion, or test. If each sensor 102 is equipped with a vibration transducer, the body part or parts that are in the incorrect position can be indicated by means of the vibration transducer.

Also, each sensor 102 can have a unique ID. Accordingly, each sensor 102 can be connected to access point 107 via, e.g., local network 108, which can be local to body suit 103 or located at a remote computer system, such as diagnostic computer 111. When connected to access point 107, sensors 102 can essentially form a local area network. When located at or on body suit 103, access point 107 can wirelessly transmit motion data, system performance data, and the like to communicate sensed data to a diagnostic computer 111. Also, communication can be enabled between camera 105 and access point 107 via, e.g., local network work 108 for communication of the foregoing types of data, digital image data, and the like.

In one embodiment, each sensor 102 may act as a master for a series of other sensors 102 serially connected thereto. In that case, a master sensor 102 is connected directly to access point 107, which includes multiple input ports for receiving data from each master sensor. In such an embodiment, the network of sensors 102 is preferably a local network where sensors 102 and access point 107 comply with the applicable communication protocols and communicate with diagnostic computer 111 via network 109, which, preferably, is a local wireless network. Access point 107 preferably includes a USB port or similar data interface when providing a wired connection to sensors 102. Likewise, sensors 102 can be powered centrally from access point 107 using the same interface that carries the data.

Accordingly, access point 107 can receive data signals from sensors 102 through a wired or wireless connection and transmit that data to a controller, which can be collocated with access point 107, located at diagnostic computer 111, or positioned at a separate location. Access point 107 can also transmit diagnostic data, control data, and system data such as, e.g., battery life information, system performance, and the like, to the controller. In doing so, access point 107 may stream all data from sensors 102 and pain value data from patient 104 to the controller, or may transmit that data in response to a call for data from the controller. Further, multiple access points 107 can be provided, each having a user-configurable IP address. This allows multiple access points 107 to be controlled by a single controller. It also allows access points 107 to be controlled via the internet from a remote diagnostic computer 111. In similar fashion multiple cameras 105 can be utilized, each also having a user-configurable IP address, and configured to communicate with one or more of access points 107.

Diagnostic computer 111 processes motion data captured by MCS 101 at one or more joints along the spine, neck, hip, knee, ankle, wrist, elbow, hand, foot, and/or shoulder. In doing so, hardware, software, and logic algorithms executing at diagnostic computer 111 can be utilized to gather and transform data associated with both macro and micro body movements of patient 104. Diagnostic computer 111 receives motion capture data from MCS 101 as well as pain value data from patient 104. Patient 104 can also be connected to a variety of monitoring devices that interface with diagnostic computer 111. Data structures comprising motion capture data are received at MCS interface 112 and data structures comprising pain value data are received via patient interface 113.

During operation of system 100, patient 104 can perform a series of selected activities, including executing a selected range of motion, or tests. As patient 104 moves, sensors 102 follow the movement substantially as though sensors 102 were rigidly attached to points on patient 104. Preferably, body suit 103 is one in which the movement of sensors 102 is limited to a predetermined range of motion, or test. The amount of movement between sensors 102 and/or the overall permitted range of motion, or test can be based on several factors, such as the type of material used in body suit 103 and the amount of force applied to body suit 103. Further, the predetermined ranges of motion, or tests are assigned to patient 104 based on, among other things, patient 104's symptoms.

Patient 104's motion can be compared to several different sets of data and for different purposes. For example, patient 104's motion can be compared to patterns modeled in software to depict idealized motion, i.e., the modeled motion of the patient without a joint disorder. This can be used to determine the characterization and severity of a joint disorder suffered by patient 104. Patient 104's motion can also be compared to patient 104's motion stored from other motion capture sessions. This can be used to evaluate patient 104's progress between capture sessions and/or the effectiveness of the current treatment regimen. Patient 104's motion can also be compared to his or her motion captured in the same session. This can be used to compare patient 104's natural motions to similar movements made with adjustments directed by a healthcare provider. Patient 104's motion can also be compared to that of another patient. This can be used to compare patient 104's motion to that of a healthy person to provide a diagnosis.

In any event, the data ultimately compared to patient 104's captured motion and pain data may be stored as sets of data in database 121. For example, if the stored data sets used for comparison relate to motion data captured from other patients, the data sets may be tagged with, or otherwise associated with, a diagnosed joint disorder. If patient 104 displays particular flaws in executing one or more selected ranges of motion, or tests and/or pain at certain positions in executing the selected ranges of motion, or tests, that information can be compiled to form a test profile. That test profile can be compared to another data set, or profile, compiled from the examination of another patient. If the flaws and pain points in patient 104's test profile match or substantially overlap with the flaws and pain points from a stored test profile relating to another patient, then the diagnosis associated with the stored test profile may then be attributed to patient 104's test profile. Once this association is made, the healthcare provider can share the diagnosis with patient 104 and develop an appropriate treatment protocol.

Further, real-time diagnostic information can be provided and displayed on graphical user interface (GUI) 131 or a or similar display in a standalone application or via a web based system using a web server. The display can incorporate without limitation 3D medical anatomical displays, biomechanical data interpretation, and interactive imaging that is needed for the diagnosis and/or treatment of patient 104. Using GUI 131, diagnostic computer 111 may provide a healthcare provider with a diagnostic solution that includes visual and verbal guidance instructions directing the steps needed for correction of the joint disorder. Accordingly, the use of the logic algorithms can help to identify dysfunctions, and can in many cases identify probable causes of the dysfunction. In some embodiments, the logic algorithms can, utilizing, e.g., an interactive tool presented to a user via GUI 131, visually and verbally guide the healthcare provider in a step by step process to diagnose patient 104's dysfunctions.

Diagnostic computer 111 can be used to implement processing functionality for various aspects of the current disclosure. Diagnostic computer 111 may comprise, e.g., a user device such as a desktop, mobile device, a mainframe, server, or any other type of special or general purpose computing device as may be desirable or appropriate for the given application or environment. Diagnostic computer 111 can include one or more processors, such as a processor 114. Processor 114 can be implemented using a general or special purpose processing engine such as, for example, a microprocessor, microcontroller or other control logic to specifically execute the diagnostic operations described herein. In this example, processor 114 is connected to a bus 116 or other communication medium.

Diagnostic computer 111 can also include a main memory 115, such as random access memory (RAM) or other dynamic memory, for storing information and instructions to be executed by processor 114. Main memory 115 also may be used for storing temporary variables or other intermediate information during execution of instructions to be executed by processor 114. Diagnostic computer 111 may likewise include a read only memory (“ROM”) or other static storage device (not shown) coupled to bus 116 for storing static information and instructions for processor 114.

Diagnostic computer 111 may also include media drive 117 and removable storage media 118. Media drive 117 may include a drive or other mechanism to support fixed or removable storage media, such as a hard disk drive, a floppy disk drive, a magnetic tape drive, an optical disk drive, a CD or DVD drive (R or RW), or other removable or fixed media drive. Storage media 118 may include, for example, a hard disk, floppy disk, magnetic tape, optical disk, CD or DVD, or other fixed or removable medium that is read by and written to by media drive 117. As these examples illustrate, the storage media 118 may include a computer-readable storage medium having stored therein particular computer software or data.

Diagnostic computer 111 can include other similar instrumentalities for allowing computer programs or other instructions or data to be loaded into diagnostic computer 111. Such instrumentalities may include, for example, a removable storage unit, and an interface, such as a program cartridge and cartridge interface, a removable memory (for example, a flash memory or other removable memory module) and memory slot, and other removable storage units that allow software and data to be transferred from the storage media 118 to diagnostic computer 111.

Diagnostic computer 111 can also include communications interfaces 112 and 113. Communications interfaces 112 and 113 can be used to allow software and data to be transferred between diagnostic computer 111 and external devices. Examples of communications interfaces 112 and 113 can include a modem, a network interface (such as an Ethernet or other NIC card), a communications port (such as for example, a USB port), a PCMCIA slot and card, etc. Software and data transferred via communications interfaces 112 and 113 are in the form of signals which can be electronic, electromagnetic, optical, or other signals capable of being received by communications interfaces 112 and 113. These signals are provided to communications interfaces 112 and 113 via a channel 119. Channel 119 may carry signals and may be implemented using a wireless medium, wire or cable, fiber optics, or other communications medium. Some examples of a channel include a phone line, a cellular phone link, an RF link, a network interface, a local or wide area network, and other communications channels. Essentially any physical layer connectivity and communications protocol may be employed. In some embodiments, signals are conducted wirelessly, and may employ any suitable carrier frequency, modulation scheme and protocol, such as BlueTooth, ZigBee, CDMA, GSM and the like. Data may be stored and accessed in any manner known to the art, including local storage, remote servers and cloud computing.

According to one embodiment, the components of diagnostic computer 111 are distributed. In such an embodiment, GUI 131 may be implemented on a user's mobile device or the like and functionality may be provided by an application installed on the user's mobile device. Information may be shared across system 100 as one user's mobile device communicates information with other components, including database 121 and mobile devices associated with other users. Such an embodiment is advantageous because it allows one healthcare provider to access information relating to patient 104 and share that information with patient 104 and/or other healthcare providers, while requiring only mobile devices. In such embodiments, information is encrypted to ensure privacy is protected and system 100 complies with applicable regulatory provisions. Naturally, each healthcare provider and patient 104 may be required to submit validation credentials to access the information provided by system 100.

The terms “computer program product” and “computer-readable storage medium” may be used generally to refer to media. These and other forms of computer-readable storage media may be involved in providing one or more sequences of one or more instructions to processor 114 for execution. Such instructions, generally referred to as “computer program code” (which may be grouped in the form of computer programs or other groupings), when executed, enable the diagnostic computer 111 to perform features or functions of embodiments of the current technology.

In an embodiment where the elements are implemented using software, the software may be stored in a computer-readable storage medium and loaded into diagnostic computer 111 using, e.g., removable storage drive 118 or communications interfaces 112 and 113. The control logic (in this example, software instructions or computer program code), when executed by the processor 114, causes the processor 114 to perform the functions of the technology as described herein.

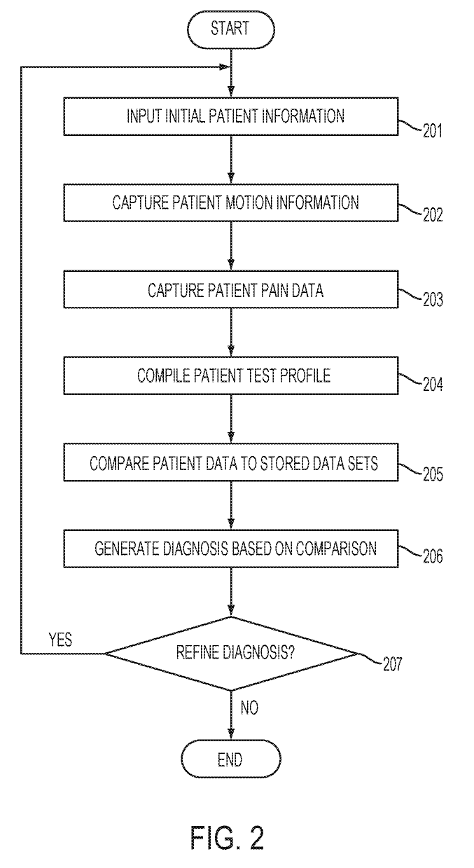

FIG. 2 is a flow chart illustrating steps in a joint disorder diagnostic method according to one embodiment. Joint disorder diagnostic method 200 begins with an assessment of the patient's information. At step 201, initial data relating to the patient is input to a diagnostic computer, such as, e.g., diagnostic computer 111 illustrated at FIG. 1. The initial data may comprise patient height, weight, sex, age, body mass index (BMI), body fat percentage, appendage circumference, and the like. Also, additional information may be entered including general medical history, medical history associated with one or more problematic joints, current treatment regimens, and imaging information (e.g., MRI information). This information can be considered as part of the diagnostic protocol.

Prior to or concurrent with step 201, a patient, such as patient 104 described with respect to FIG. 1, is screened for joint pain (e.g., posterior hip pain). Patients that exhibit joint pain will be physically examined prior to testing and, in some instances, may be excluded where it is determined the patient requires a follow-up evaluation, suffers from chronic pain and/or multi-level pathologies, has a pacemaker or metal implant, or is pregnant. Once a patient is qualified, participation requirements and the like will be explained to the patient.

At step 202, the patient's motion is monitored. Monitoring the patient involves putting the patient through one or more selected ranges of motion, or tests and evaluating the problematic joint during those ranges of motion, or tests. That is, the patient executes selected biomechanical movements designed to provide a meaningful picture of the functional biomechanics of the patient and/or possible anomalies in the patient's biomechanical/functional range of motion, or test.

By way of example, where a patient's hip joint is to be evaluated and diagnosed according to described concepts, sensors, such as sensors 102 illustrated in FIG. 1, can be positioned on the patient prior by the primary investigator. According to an embodiment, three (3) sensors can be placed in standardized locations on the femur and sacrum where, e.g., one (1) sensor can be placed on the anteromedial mid femur of the left leg, one (1) sensor can be placed on the anteromedial mid femur of the right leg, and one (1) sensor can be placed on the sacrum. Consistent with the foregoing description, the sensors can be attached to the patient using a variety of mechanisms to prevent undesired movement during physical examination.

At step 203, pain data is received from the patient as the patient executes the ranges of motion, or tests. By way of an input device, such as wireless transmitter, the patient transmits a signal upon a feeling of pain. These “pain points” can be considered as part of the diagnostic protocol. Overlaying the different positions or ranges of positions at which the patient experiences pain with the captured motion information provides further meaning to the patient's likely joint disorder. In one embodiment, the occurrence of pain is considered as a binary input, i.e., where a value of 1 represents pain while a value of 0 is maintained when no indication of pain is received from the patient. In another embodiment, a range of pain values is considered in the diagnostic proposal. By way of example, a value of 0.25 can be assigned to a position or range of positions where a patient experiences a small amount of pain while a value of 0.75 can be assigned to a position or range of positions where the patient experience significant pain.

At step 204, a patient test profile is optionally created from the captured motion data and the captured pain value data. The profile may be compiled by hardware and software residing at, e.g., diagnostic computer 111 illustrated at FIG. 1.

At step 205, a comparison is performed between the patient's captured motion data and pain data and stored data sets. This may be done by comparing the data in its raw form, or by comparing the data as compiled in a patient test profile, as described with respect to preceding optional step 204. Further, the inclusion of pain data is optional. So, in the event pain data is captured from the patient, but not include in the stored data sets, that information may be omitted from the comparison.

As discussed, the patient's motion can be compared to several different sets of data and for different purposes. The patient's motion can be compared to patterns modeled in software to depict idealized motion, i.e., the modeled motion of the patient without a joint disorder. This can be used to determine the characterization and severity of a joint disorder suffered by the patient. The patient's motion can also be compared to his or her motion stored from other motion capture sessions. This can be used to evaluate the patient's progress between capture sessions and/or the effectiveness of the current treatment regimen. The patient's motion can also be compared to his or her motion captured in the same session. This can be used to compare the patient's natural motions to similar movements made with adjustments directed by a healthcare provider. The patient's motion can also be compared to that of another patient. This can be used to compare the patient's motion to that of a healthy person to provide a diagnosis. When the stored data sets used for comparison relate to motion data captured from other patients, the data sets may be tagged with, or otherwise associated with, a diagnosed joint disorder.

At step 206, based upon the comparison, a diagnosis (or probable diagnosis) is provided. By way of example, if the patient displays particular flaws in executing one or more selected ranges of motion, or tests and/or pain at certain positions in executing the selected ranges of motion, or tests, that information can be compiled to form a test profile. That test profile can be compared to another data set, or profile, compiled from the examination of another patient. If the flaws and pain points in the patient's test profile match or substantially overlap with the flaws and pain points from a stored test profile relating to another patient, then the diagnosis associated with the stored test profile may then be attributed to the patient's test profile. Once this association is made, the healthcare provider can share the diagnosis with the patient and develop an appropriate treatment protocol.

At step 207, a refinement of the initial diagnosis can be performed. In that case, once motion data has been acquired, the logic software can analyze the patient's range of motion, or test and associated pain points and instruct the patient to perform further movements and activities based on the correlated data and interpretation of the patient's range of motion, or test and associated pain points. Accordingly, another dynamic phase of motion capture can be performed. For example, the patient can repeat selected ranges of motion, or tests that have proven difficult or painful. The patient can indicate pain during these activities and the modeling software can consider the received indications of pain in its diagnostic protocol.

### Example

Consider the case where a patient is suspected of suffering from a hip joint disorder. The patient will undergo a preliminary evaluation in which vital information such as BMI, medical information such as medical history, and imaging information such as CT images will be input to the diagnostic computer for consideration in the diagnostic protocol.

CT images are input to the diagnostic computer to develop a bony mesh model, which allows a user to view images that realistically portray the patient's motion. Incorporating software that uses patient MRI/CT images further enhances the images of the patient by depicting his or her true anatomy.

The patient will then be fitted with a body suit about the hip joint and move through a battery of tests thought to enable diagnosis of hip joint disorders. In this exemplary embodiment, sensors will be placed at least about the (1) Sacrum, (2) anteromedial location of mid left femur, and (3) anteromedial location of mid right femur.

Sensors placed on the body suit are capable of measuring six degrees of freedom. Raw data transmitted from the sensors will consist of quantitative measures of, e.g., distance and angular rotation. Accelerometers and other inertial devices provide information relating to acceleration, velocity, and limits on the patient's dynamic movements. The patient's motion can be captured by motion capture software such as MotionMonitor™, manufactured by Innovative Sports Training, Inc. of Chicago, Ill. Concurrently, the patient will transmit data structures indicative of pain level experience during the tests. The data structures may be transmitted via a wired or wireless interface and communication protocol.

Each test comprises one or more selected ranges of motion or tests that are accepted as useful in diagnosing one or more disorders. Each of the selected ranges of motion and specific tests may be executed in a particular order. The order in which the tests are to be completed may be established beforehand as part of the diagnostic protocol. Further, in some cases, it may preferable for the patient to complete all ranges of motion or tests in the particular order for one side of the body (e.g., the left side), and then complete all ranges of motion or tests in the same order for the other side of the body (e.g., the right side). In other cases, it may be preferable for the patient to complete one range of motion or test for each side of the body before moving on to the next range of motion or test in the particular order.

Preferably, the patient completes tests in the following order: (1^{st}) standing, (2^{nd}) seated, (3^{rd}) supine, (4^{th}), lateral and (5^{th}) prone. In doing so, according to the exemplary embodiment relating to diagnosing a hip and/or spine joint disorder, the patient undergoes one or more of the following examinations:

STANDING EXAMINATION comprising: Normal Gait, Long Stride, Short Stride, and Trendelenberg test;

SEATED EXAMINATION comprising: Straight Leg Raise, Internal Rotation, External Rotation, Piriformis Stretch Test, Active Knee Flexion Test Against Resistance;

SUPINE EXAMINATION comprising: Thomas Test R+− L+−, FADDIR R+− L+−, DIRI R+− L+−, DEXRIT R+− L+−, −Posterior Rim Impingement Test R+− L+−, Apprehension R+− L+−, Tinel-Femoral Nerve R+− L+−, FABER/Patrick Test R+− L+−, Straight Leg Against Resistance/Stitchfield Test R+− L+−, Log Roll R+− L+−, Heel Strike R+− L+−;

LATERAL EXAMINATION comprising: Lateral Rim Impingement Test R+− L+−, FADDIR R+− L+−; and

PRONE EXAMINATION comprising: Ely R+− L+−, Craig (degrees anteversion).

More specifically, a medical professional may utilize the examinations as comprised in a standard hip/spine intake from as shown in Table 1. These examinations include, in addition to range of motion examinations, other useful information, i.e., neurological findings, patient history, circulation (blood and lymphatics), etc. Thus, the information obtain from the below Table 1 can distinguish between a hip joint disorder and a spine related disorder, thereby enabling the healthcare provider to determine the appreciate course of treatment.

Once the data is received, the diagnostic computer executes the diagnostic protocol. According to this exemplary embodiment, the protocol includes a binary system of positive (pain) and negative (no pain) values, in addition to specific limits for each test that correspond to each level of the physical examination. Upon painful stimulation, the patient transmits a signal indicative of pain. The algorithm will process the recorded data based on the positive and negative values to provide a possible diagnosis. The underlying process overlays the pain points received from the patient with other considerations. As mentioned, the captured motion data and pain data may be compiled to form a test profile. Data fields within the test profile may be compared to other sets of stored data to associate or tag the patient's test profile with a diagnosis. For example, the diagnostic computer may determine the patient suffers from:

Loss of IR+FADDIR+DIRI+DEXRIT. Based on the comparison to data sets comprising similar data fields (correlated to symptoms), the diagnostic protocol yields “Labral Tear (anterior)” as the diagnosis.

According to this exemplary embodiment, the following definitions would be used: “IR” refers to internal rotation of the hip joint as known by one of skill in the art; “ER” refers to external rotation of the hip joint as known by one of skill in the art; “Trendelenberg test” refers to the Single Leg Stance Phase Test, i.e., where the patient stands with feet shoulder width apart and then brings one leg forward by flexing the leg to 45 degrees at the hip and 45 degrees at the knee, the single leg stance phase position is held for six seconds testing the contralateral hip abductor musculature and neural loop proprioception, a pelvic tilt greater than 2 cm constitutes a positive shift; “Thomas Test” refers to the Hip Flexion Contracture Test, i.e., the patient extends and relaxes one leg down toward the table, while holding the contralateral leg in full flexion, the inability of the thigh to reach the table shows a hip flexion contracture; “FADDIR” refers to Flexion Adduction Internal Rotation, i.e., the examiner holds the monitoring hand about the superior aspect of the hip with the lower leg cradled on the forearm with the knee upon the hand, the hip is then brought into flexion, adduction, and internal rotation, a complaint from the patient during the test signifies a positive result for this test; “DIRI” refers to Dynamic Internal Rotatory Impingement Test, the patient is in the supine position and directed to hold the non-affected leg in flexion beyond 90 degrees, the examined hip is then brought into 90 degrees of flexion, or beyond, and is passively taken through a wide arc of adduction and IR, a complaint from the patient during the test signifies a positive result for this test; “DEXRIT” refers to Dynamic External Rotatory Impingement Test, i.e., with the patient in the supine position, the patient is instructed to hold the non-affected leg beyond 90 degrees, the examined hip is then brought into 90 degrees of flexion, or beyond, and is passively taken through a wide are of abduction and ER, a complaint of pain during the test signifies a positive result; “FABER”, also known as the Patrick Test, refers to Flexion Abduction External Rotation, a complaint of pain during the test signifies a positive result for this test; “Tinel of the femoral nerve” refers to a test in which a Heel Strike is carried out by striking the heel, a complaint of pain during this test signifies a positive result; “Log Roll” refers to the PSR test, i.e., the examiner passively moves the patients' leg in IR and ER of the femur, a complaint of pain during this test signifies a positive result; Lateral Rim Impingement Test” refers a test in the supine position, i.e., with the hip passively abducted and externally rotated, the examiner cradles the patient's lower leg with one arm and monitors the hip joint with the opposing hand, the examiner passively brings the affected hip through a wide arc from flexion to extension in continuous abduction while externally rotating the hip, a complaint of pain during this test signifies a positive result; “Craig Test” refers to the Femoral Anteversion Test, i.e., with the patient in the prone position, the knee is flexed to 90 degree and the examiner manually rotates the leg while palpating the greater trochanter so that it protrudes most laterally, femoral version is assessed by noting the angle between the axis of the tibia and an imaginary vertical line; “Ely Test” refers to the Rectus Contracture Test, i.e., with the patient in the prone position, the lower extremity is flexed toward the gluteus maximus, a rise of the pelvis or restriction of hip flexion signifies a positive result.

According to described embodiments, the diagnostic protocol may utilize the foregoing in comparing the patient's motion and pain data to other data sets, which themselves are tagged or otherwise associated with a diagnosis. Based on the comparison, the patient's test profile may be tagged with a diagnosis associated with the data set most closely matching the patient's test profile. In this exemplary embodiment, a hip joint diagnosis is provided as follows:

TABLE 2
Hip Joint Diagnosis

| Painful Physical | |
|---|---|
| Examination Movements | Diagnosis |
| +Loss of IR, +FADDIR, +DIRI, | Labral tear (anterior) |
| +DEXRIT | |
| +Loss of IR, +LRI, +FABER, | Labral tear (superior) |
| +DEXRIT | |
| +Loss of IR, +PRI, +DEXRIT | Labral tear (posterior) |
| +Loss of IR, +FADDIR, +DIRI, | FAI CAM |
| +DEXRIT | |
| +Loss of IR, +FADDIR, +DIRI, | FAI pincer (anterior) |
| +DEXRIT | |
| +LRI, +Decrease in ER, +FABER, | FAI pincer (lateral) |
| +DEXRIT | |
| +Decrease in ER, +PRI, +FABER | FAI pincer (posterior) |
| +Craig test, +ABDEER | Hip Instability, Osseous |
| +ABDEER, +PSR, +FABER, | Hip Instability, Ligamentous |
| +PRI, +Apprehension | |
| +Contracture Test, +Passive | Hip Instability, Musculotendenous |
| Adduction Test | |
| +Decrease IR/ER | Chondral Lesion, Acute |
| +FADDIR, +DIRI, +LRI, +FABER, | Chondral Lesion, Chronic |
| +PRI, +DEXRIT, +Scour | |
| +Restricted ROM, +Palpation, | GT Pain |
| +Passive Adduction Test | |
| +Fan test | Snapping Hip, psoas |
| +Bicycle test | Snapping Hip, IT band |

According to further embodiments, the diagnostic protocol compares the patient's motion and pain data to other data sets, which themselves are tagged or otherwise associated with a diagnosis, to diagnose other joint disorders. Again, the patient's test profile may be tagged with a diagnosis associated with the data set most closely matching the patient's test profile. In this exemplary embodiment, a knee disorder diagnosis is provided as follows:

TABLE 3
Knee Disorder Diagnosis

| Physical Exam | Diagnosis |
|---|---|
| +Patella Grind Test | Patellofemoral pathology |
| +Patella Apprehension Test | Patella subluxation/dislocation |
| +Patella Tilt Test | >15° = Lax <0° + tight lateral constraint |
| +McMurray's Medial | Medial Meniscus Tear |
| Meniscus Test | |
| +McMurray's Lateral | Lateral Meniscus Tear |
| Meniscus Test | |
| +Apley's Grind Test | Meniscus Tear |
| +Valgus Stress Test | MCL/LCL laxity (grade I-III) |
| +Varus Stress Test | MCL/LCL and PCL |
| +Anterior Drawer Test | ACL injury |
| +Lachman Test | ACL injury |
| +Posterolateral Drawer Test | PLC injury |
| +McIntosh Pivot Shift Test | |

According to this exemplary embodiment, the following definitions would be useful for knee diagnosis: Pattella Grind refers to a test in which the patient fully flexes and extends the knee while the examiner has a hand on the front of the knee, i.e., a positive diagnosis is if crepitus or catching is felt; Pattella Apprehension Test refers to a test in which the patella is pushed in a lateral direction with the knee flexed, i.e., positive diagnosis is noted if the patient cannot flex the knee while pressure is applied; Patella Tilt refers to a test in which the examiner holds the patella between the thumb and index finger, i.e., there should only be a slight difference in the horizontal plane; McMurray Medial Meniscus Test refers to a test in which the examiner palpates the medial joint line while the leg is in full flexion, i.e., the examiner then rotates the foot externally while abducting the leg, a recreation of pain and a “click” signifies a positive diagnosis; Murray's Lateral Meniscus Test refers to a test in which the examiner palpates the medial joint line while the leg is in full flexion, i.e., the examiner then rotates the foot internally while adducting the leg, a recreation of pain and a “click” signifies a positive diagnosis; Apley Grind Test refers to a test in which the examiner rotates the foot from external rotation, with the knee fully flexed, to internal rotation, with the knee fully extended, while the patient is prone, i.e., the examiner then applies compression to the sole of the foot during this motion, recreation of pain is a positive diagnosis; Valgus Stress Test refers to a test in which the examiner applies a quick valgus stress by abducting the lower leg, i.e., positive diagnosis for this test is if the medial joint line opens; Varus Stress Test refers to a test in which the examiner applies a quick various stress while the knee is in full extension and at 30°, i.e., a positive diagnosis for this test is if abnormal lateral joint opening is observed; Anterior Drawer Test refers to a test in which the examiner places their thumbs on the medial and lateral joint sulci while the symptomatic knee is flexed in 90° with the foot on the table, i.e., a positive diagnosis for this test is production of anterior translation; Lachman Test refers to a test in which the examiner uses one hand to lift the tibia in a forward direction while the other hand stabilizes the femur, i.e., positive diagnosis is produced if there is a 5-10 mm increase in anterior tibial movement from the contralateral side; Posterolateral Drawer Test refers to a test in which the examiner flexes the patients' knee to 20° while the patient lifts their heel off of the examination table, i.e., positive diagnosis for this procedure is if there is posterior translation of the tibia in relation to the femur; McIntosh Pivot Shift Test refers to a test in which subluxation of the lateral tibial plateau is observed; and Valgus stress is applied to the leg while it is in extension and internal rotation.

According to further embodiments, the diagnostic protocol compares the patient's motion and pain data to other data sets, which themselves are tagged or otherwise associated with a diagnosis, to diagnose other joint disorders. Again, the patient's test profile may be tagged with a diagnosis associated with the data set most closely matching the patient's test profile. In this exemplary embodiment, a shoulder disorder diagnosis is provided as follows:

TABLE 4
Shoulder Disorder Diagnosis

| Painful Physical Examination Movements | Diagnosis |
|---|---|
| +Neer's Impingement Sign | Subacromial impingement |
| +Hawkin's Impingement Sign | Subacromial impingement |
| +Supraspinatus Stress Test | Supraspinatus tendon tear/ |
| | Rotator Cuff |
| +Drop Arm Test | Rotator Cuff tear |
| +Lift-off Test | Possible Subscapularis tear |
| +Apprehension Test | Shoulder Instability |
| +Relocation Test | Shoulder Instability |
| +Load and Shift Test | Shoulder Instability |
| +Sulcus Sign | Inferior Shoulder Laxity |
| +Jerk Test | Posterior Instability |
| +O'Brien's Test | Superior Labral Anterior and |
| | Posterior Tears |
| +Cross-body Adduction Test | AC joint degeneration |

According to this exemplary embodiment, the following definitions would be useful for shoulder diagnosis: Neer's Impingement Sign refers to the elevation of the arm in a passive and forward direction, i.e., this motion may cause the supraspinatus tendon to be impinged underneath the coracoacromial arch, a recreation of pain signifies a positive diagnosis for this test; Hawkin's Impingement Sign refers to a test in which the shoulder is flexed to 90° with adduction and internal rotation, i.e., recreation of pain signifies a positive diagnosis for this test; Supraspinatus Stress Test refers to a test for a supraspinatus tendon tear, i.e., the test is performed with the arm in forward flexion and abduction with resistance and a recreation of pain signifies a positive diagnosis for this test; Drop Arm Test refers to a test to detect more significant rotator cuff tears, i.e., the patient is asked to abduct the shoulder to 90° and then move lower, a recreation of pain signifies a positive diagnosis for this test; Lift-Off Test refers to a test in which the shoulder the patient places their hand over the abdomen and gently pushes, i.e., if the patient cannot keep the elbow in front of the midaxillary line a positive diagnosis is signified for this test; Apprehension Test refers to a test in which the shoulder is placed in external rotation and abduction to produce an unstable placement, i.e., a positive diagnosis for this test is if the patients resists the motion; Relocation Test refers to a test in which a posterior force is applied to the posterior humerus while the patient is in a position of apprehension. Load and Shift Test refers to a test in which the patient is seated and has the arm adducted, i.e., the examiner moves the humerus anterior and posterior and grades the amount of movement; Sulcus Sign refers to a test in which the patient has the arms by the side and the examiner places traction in a downward movement to produce a space between the humeral head and acromion, i.e., the size of the space can then be examined; Jerk Test refers to a test to recreate posterior subluxation by placing force on the arm which is forward flexed and in adduction, i.e., a noise may be heard or felt when the arm in the coronal plane; O'brien's Test refers to a test used to examine labral pathology, with pain signifying a positive diagnosis for this test; Cross-Body Adduction Test refers to a test in which the examiner palpates the AC joint while moving the patients arm, which is in adduction, across the chest, i.e., recreation of pain signifies a positive diagnosis for this test.

### Example

Consistent with the foregoing disclosure and according to other aspects of the inventive concepts, a posterior hip pain testing sequence can be performed, which is designed to assess the following pathologies: 1) Ischiofemoral Impingement 2) Hip Spine Effect 3) Deep Gluteal Syndrome 4) Hamstrings Syndrome. According to such an embodiment, some or all of seven (7) patient movements will be performed assessing the patient's movement during gait, in the lateral, and seated positions, respective of each test. These movements, which have been proven to be clinically useful for accurate diagnosis for each pathology, include:

Ischiofemoral Impingement:
1. long stride walk (hip extension) (gait)
2. ischiofemoral impingement test (lateral)

Hip Spine Effect:
3. hip spine test (lateral)

Deep Gluteal Syndrome:
4. active piriformis test (lateral)
5. passive piriformis test (seated)

Hamstrings Avulsion:
6. active 30 degree and active 90 degree hamstrings test (seated)
7. long stride walk (hip flexion) (gait)

In this instance, the patient is fitted with the motion capture suit following the physical examination. All study personnel will be blinded to the primary investigator assessment. Two separate system preference files will be created that correspond to the left or right hip, based on the patient's complaint, and the complaint hip will be examined at time of study.

To optimize motion capture, the patient will face the appropriate direction based on electromagnetic field calibration. The patient's body will be digitized using the 3D Motion Capture software and a “digitization pointer” that has been calibrated and saved in system preferences. According to an embodiment, the digitization procedure consists of placing a “digitization pointer” at the assigned anatomic landmarks:

1. left ASIS

2. right ASIA

3. L5-S1

4. left knee, lateral

5. left knee, medial

6. right knee, lateral

7. right knee, medial.

Using data communicated from the sensors and related system components, a user will assess the patient using the protocols discussed herein. In doing so, pain data provided by the patient to system inputs (e.g., using a push button or the like) can be integrated with the 3D Motion Capture data. The 3D Motion Capture system will record orthopedic angles including: hip flexion/extension, hip abduction/adduction, hip internal/external rotation, pelvic tilt, pelvic side bending, and pelvic rotation. The push button, or “pain data,” is also recorded in the software and/or hardware.

Consistent with the foregoing discussion, raw patient data will be analyzed using an algorithm designed to assess both (1) pain response data, and (2) orthopedic angles recorded during examination. In this instance, an algorithm can be executed using the following conditions for each of the four (4) pathologies:

1. Ischiofemoral Impingement
a. Pain only with Long Stride Walk=94% sensitivity
b. Pain only with Ischiofemoral Impingement test=82% sensitivity
c. Pain with both Long Stride Walk and Ischiofemoral Impingement test=76% sensitivity for diagnosis of Ischiofemoral Impingement

2. Hip Spine Effect
a. Hip extension less than 10 degrees and Pelvic tilt greater than 3 degrees=Positive Hip Spine Effect diagnosis

3. Deep Gluteal Syndrome
a. Pain only with Active Piriformis test=78% sensitivity
b. Pain only with Passive Piriformis test=52% sensitivity
c. Pain with both Active Piriformis test and Passive Piriformis test=91% sensitivity for diagnosis of Deep Gluteal Syndrome

4. Hamstrings Syndrome
a. Pain only with Active 30°/90° Hamstrings test=72% sensitivity
b. Pain only with Long Stride Walk=55% sensitivity
c. Pain with both Active 30°/90° Hamstrings test and Long Stride Walk=95% sensitivity for diagnosis of Hamstrings Syndrome

The system processes the data for each movement and assigns a result based on detected positive correlations. The sensitivity is based on accepted data for each movement, and reflects the ability to correctly detect the true positive rate of the movement for each pathology. The output can then be compared to the primary investigator's assessment.

Experimental results demonstrate that the inventive concepts perform extremely well in automating the diagnosis of joint disorders. These favorable results are seen at the following table, which specifies the level of agreement between the automated diagnoses achieved by the concepts described herein and a manual diagnosis. As seen, after evaluating the results of motion tests and pain data, the automated diagnoses system achieved an accuracy rate of ninety-three (93) percent:

| | | Ischiofemoral | | Hip Spine | Deep Gluteal | | Hamstring | | |
|---|---|---|---|---|---|---|---|---|---|
| | | Impingement | | Effect | Syndrome | | Syndrome | | |
| | | | IFI | Hip Spine | Active | Passive | Active | | |
| Subject | Investigator | LSWext | test | Test | Piriformis | Piriformis | 30/90 | LSWflex | % Correct |
| 001DG | PI | X | X | X | X | X | X | X | 100% |
| | 3DMC | X | X | X | X | X | X | X | |
| 003CS | PI | ✓ | X | X | ✓ | ✓ | X | ✓ | 86% |
| | 3DMC | ✓ | ✓ | X | ✓ | ✓ | X | ✓ | |
| 004SG | PI | X | ✓ | ✓ | X | X | X | ✓ | 100% |
| | 3DMC | X | ✓ | ✓ | X | X | X | ✓ | |
| 005MI | PI | ✓ | ✓ | ✓ | X | ✓ | X | X | 86% |
| | 3DMC | ✓ | ✓ | ✓ | ✓ | ✓ | X | X | |

| | |
|---|---|
| Avg % correct | 93 |

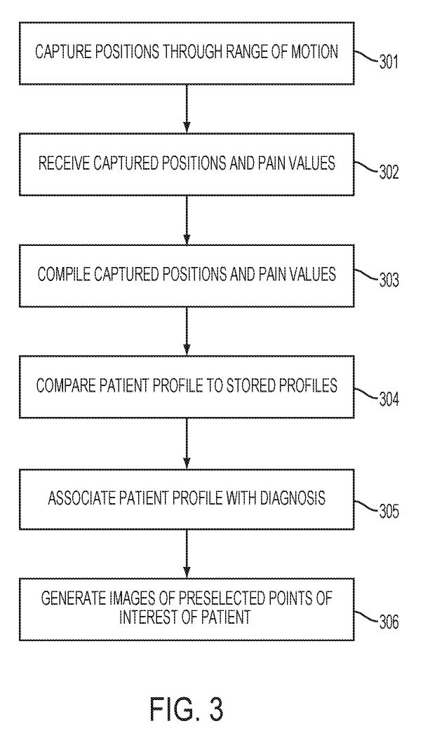

FIG. 3 illustrates functional blocks executed to perform joint disorder diagnosis method according to the concepts described herein. Specifically, FIG. 3 illustrates functional blocks executed by, e.g., system components illustrated at FIG. 1, such as diagnostic computer 111. At step 301, sensors attached to a patient capture positions for one or more preselected points of interest of a test patient through a preselected range of motion, or test. At step 302, a diagnostic computer in communication with the sensors receives the captured positions and a pain value associated with the captured positions.

At step 303, the diagnostic computer compiles the captured positions and the pain values associated with the captured positions to generate a test patient profile. Generating the test patient profile may also comprise correlating a binary pain value with each of a plurality of captured positions through the preselected range of motion, or test. Generating the test patient profile may further comprise correlating additional information, e.g., the body mass index (BMI) of the test patient, with the binary pain values and the captured positions.

At step 304, the diagnostic computer compares the test patient profile to one or more stored profiles. The one or more stored profiles comprises positions for the one or more preselected points of interest of another test patient through the preselected range of motion, or test and a pain value associated with the positions. Each stored profile is associated with a diagnosis.

At step 305, the test patient profile is associated with a diagnosis, which is associated with the one or more stored profiles that matches the test patient profile.

At step 306, the diagnostic computer generates images of the preselected points of interest of the test patient with markers indicative of the probable diagnosis. The one or more images comprise visual representations of the captured positions and pain values associated with the captured positions through the preselected range of motion, or test. This information may be overlaid with the probably diagnosis and its symptoms, to visualize the closeness of the match.

Further, in some embodiments, the preselected points of interest comprise a hip joint. Also, the preselected range of motion, or test may comprise at least one of: internal rotation of the hip joint; external rotation of the hip joint; hip flexion; and pelvic tilt.

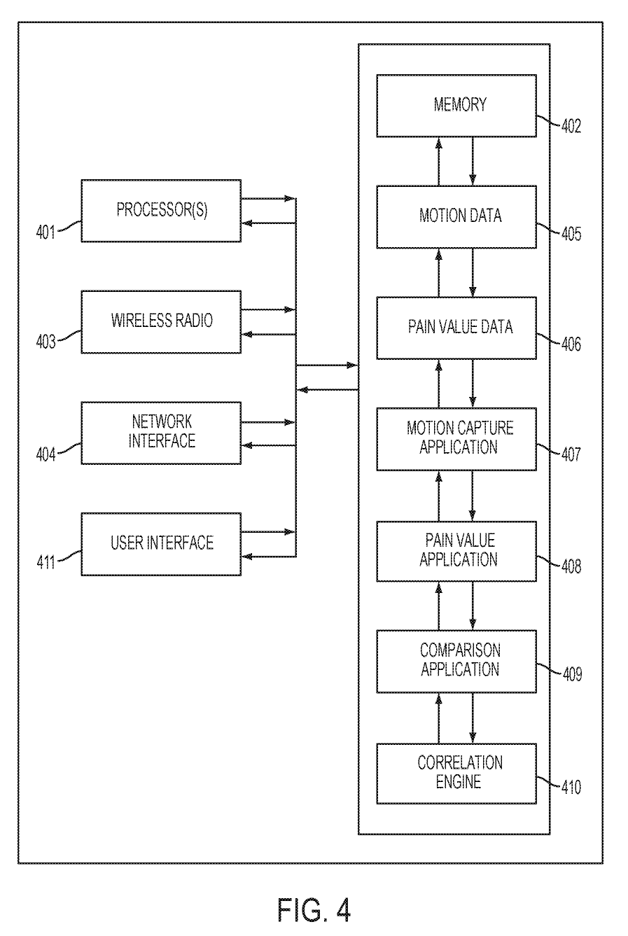

FIG. 4 illustrates, in more detail, components of an apparatus that enables joint disorder diagnosis according to the concepts described herein. Referring to FIG. 4, diagnostic module 400 may correspond to diagnostic computer 111 illustrated in FIG. 1. Components of module 400 may comprise hardware, software, firmware, program code, or other logic (for example, ASIC, FPGA, etc.), as may be operable to provide the functions described herein. Module 400 comprises components that, when executing operations described herein, effectuate joint disorder diagnosis mechanisms. Each of these components can be separate from, or integral with, module 400.

The functionality and operations of module 400 are controlled and executed through processor(s) 401 and specialized software executing thereon. Processor(s) 401 may include one or more core processors, central processing units (CPUs), graphical processing units (GPUs), math co-processors, and the like. Processor(s) 401 execute program logic, whether implemented through software stored in a memory 402 or in firmware in which logic is integrated directly into integrated circuit components. Module 400 may communicate wirelessly with other system components. Processor(s) 401 execute program logic, whether implemented through software stored in a memory 402 or in firmware in which logic is integrated directly into integrated circuit components. Module 400 may communicate wirelessly with other system components, such as multiple nodes comprising MCS 101, patient 104, and access point 107, through various radios, such as wireless radio 403, such as one or more of wireless wide area network (WWAN) radios and wireless local area network (WLAN) radios. If a WWAN radio is included as one of the radios in wireless radio 403, communication would generally be allowed over a long range wireless communication network such as 3G, 4G, LTE, and the like. Various WLAN radios, such as WIFI™ radios, BLUETOOTH® radios, and the like, would allow communication over a shorter range. Module 400 may also provide communication and network access through a wired connection with network interface 404. The wired connection may connect to the public-switched telephone network (PSTN), or other communication network, in order to connect to the Internet or other accessible communication network.

Module 400 may include memory 402 that stores signals that are used to transmit and receive data and control data, including patient diagnostic data. Memory 402 may be provided as a separate component and affixed to or formed integrally with module 400. The exact location of memory 402 is not limited and memory 402 may be provided with any form of communication means to communicate with MCS 101 and module 400. Memory 402 may include any type of data storage means such as computer readable memory or the like. Preferably, memory 402 stores captured motion data 405 and pain value data 406.

Under control of processor(s) 401, program logic stored on memory 402, including motion capture application 407, pain value application 408, comparison application 409, correlation engine 410, and other applications are executed to provide the functionality of module 400, including communications, storage, computation, filtering, analysis, and correlation of motion capture data and pain value data, and transmitting data to system components to ensure joint disorder diagnosis. Various operating applications may be displayed visually to the user via user interface 411.

Motion capture application 407 extracts data structures indicative of capture positions for one or more preselected points of interest of a test patient through a preselected range of motion, or test from sensors arranged on a body suit attached to the test patient.

Pain value application 408 receives data structures indicative of pain value associated with the captured positions from the test patient.

Comparison application 409 compiles the captured positions and the pain values associated with the captured positions to generate a test patient profile. Afterward, comparison application 409 compares the test patient profile to one or more stored profiles, the one or more stored profiles comprising positions for the one or more preselected points of interest of another test patient through the preselected range of motion, or test and a pain value associated with the positions, each stored profile associated with a diagnosis.

Based on the comparing, correlation engine 410 associates the test patient profile with a diagnosis, which itself is associated with the one or more stored profiles that matches the test patient profile.

1. A joint disorder diagnostic method, the method comprising:
capturing, using sensors attached to a test patient, positions for one or more preselected points of interest of the test patient through a preselected range of motion, or test;
receiving, at one or more processors in communication with the sensors, the captured positions and a pain value associated with the captured positions;
compiling, with the one or more processors, the captured positions and the pain values associated with the captured positions to generate a test patient profile;
comparing, with the one or more processors, the test patient profile to one or more stored profiles, the one or more stored profiles comprising positions for the one or more preselected points of interest of another test patient through the preselected range of motion, or test and a pain value associated with the positions, each stored profile associated with a diagnosis; and
based on the comparing, associating the test patient profile with a diagnosis associated with the one or more stored profiles that matches the test patient profile.

2. The joint disorder diagnostic method of claim 1 further comprising:
generating one or more images of the preselected points of interest of the test patient, the one or more images comprising visual representations of the captured positions and pain values associated with the captured positions through the preselected range of motion, or test.

3. The joint disorder diagnostic method of claim 1 where generating the test patient profile comprises:
correlating a binary pain value with each of a plurality of captured positions through the preselected range of motion, or test.

4. The joint disorder diagnostic method of claim 3 where generating the test patient profile further comprises:
correlating the body mass index (BMI) of the test patient with the binary pain values and the captured positions.

5. The joint disorder diagnostic method of claim 1 where the preselected points of interest comprise a hip joint.

6. The joint disorder diagnostic method of claim 5 where the preselected range of motion, or test comprises at least one of:
internal rotation of the hip joint;
external rotation of the hip joint;
hip flexion; and
pelvic tilt.

7. A joint disorder diagnostic system, the system comprising:
sensors arranged on a body suit to capture positions for one or more preselected points of interest of a test patient through a preselected range of motion, or test;
one or more processors in communication with the sensors, the one or more processors configured to:
receive the captured positions and a pain value associated with the captured positions;
compile the captured positions and the pain values associated with the captured positions to generate a test patient profile;
compare the test patient profile to one or more stored profiles, the one or more stored profiles comprising positions for the one or more preselected points of interest of another test patient through the preselected range of motion, or test and a pain value associated with the positions, each stored profile associated with a diagnosis; and
based on the comparing, associate the test patient profile with a diagnosis associated with the one or more stored profiles that matches the test patient profile.

8. The joint disorder diagnostic system of claim 7 where the one or processors is further configured to:
generate one or more images of the preselected points of interest of the test patient, the one or more images comprising visual representations of the captured positions and pain values associated with the captured positions through the preselected range of motion, or test.

9. The joint disorder diagnostic system of claim 7 where the one or processors is further configured to:
correlate a binary pain value with each of a plurality of captured positions through the preselected range of motion, or test.

10. The joint disorder diagnostic system of claim 9 where the one or processors is further configured to:
correlate the body mass index (BMI) of the test patient with the binary pain values and the captured positions.

11. The joint disorder diagnostic system of claim 7 where the preselected points of interest comprise a hip joint.

12. The joint disorder diagnostic system of claim 11 where the preselected range of motion, or test comprises at least one of:
internal rotation of the hip joint;
external rotation of the hip joint;
hip flexion; and
pelvic tilt.

13. A joint disorder diagnostic apparatus, the apparatus comprising:
a memory; and
one or more processors in communication with the memory, the one or, the one or more processors configured to:
receive data structures indicative of capture positions for one or more preselected points of interest of a test patient through a preselected range of motion, or test from sensors arranged on a body suit attached to the test patient;
receive data structures indicative of pain value associated with the captured positions from the test patient;
compile the captured positions and the pain values associated with the captured positions to generate a test patient profile;
compare the test patient profile to one or more stored profiles, the one or more stored profiles comprising positions for the one or more preselected points of interest of another test patient through the preselected range of motion, or test and a pain value associated with the positions, each stored profile associated with a diagnosis; and
based on the comparing, associate the test patient profile with a diagnosis associated with the one or more stored profiles that matches the test patient profile.

14. The joint disorder diagnostic apparatus of claim 13 where the one or processors is further configured to:
generate one or more images of the preselected points of interest of the test patient, the one or more images comprising visual representations of the captured positions and pain values associated with the captured positions through the preselected range of motion, or test.

15. The joint disorder diagnostic apparatus of claim 13 where the one or processors is further configured to:
correlate a binary pain value with each of a plurality of captured positions through the preselected range of motion, or test.

16. The joint disorder diagnostic apparatus of claim 15 where the one or processors is further configured to:
correlate the body mass index (BMI) of the test patient with the binary pain values and the captured positions.

17. The joint disorder diagnostic apparatus of claim 13 where the preselected points of interest comprise a hip joint.

18. The joint disorder diagnostic apparatus of claim 17 where the preselected range of motion, or test comprises at least one of:
internal rotation of the hip joint;
external rotation of the hip joint;
hip flexion; and
pelvic tilt.
